(19) United States
(12) Patent Application Publication
(10) Pub. No.: US 2021/0069102 A1
(43) Pub. Date: Mar. 11, 2021

(54) METHODS FOR PREVENTION AND TREATMENT OF UROGENITAL ATROPHY OF MENOPAUSE BY CONTACT VASODILATORS

(71) Applicant: Assa Weinberg, Los Angeles, CA (US)

(72) Inventor: Assa Weinberg, Los Angeles, CA (US)

(21) Appl. No.: 16/952,845

(22) Filed: Nov. 19, 2020

Related U.S. Application Data
(63) Continuation of application No. PCT/US2019/033120, filed on May 20, 2019.
(60) Provisional application No. 62/675,144, filed on May 22, 2018.
(60) Provisional application No. 62/675,776, filed on May 24, 2018.
(60) Provisional application No. 62/676,239, filed on May 24, 2018.

(51) Int. Cl. A61K 9/00; A61K 31/50; A61K 31/41; A61K 31/4418; A61K 31/401; A61K 31/4178; A61K 31/4184; A61P 15/02; A61K 31/277; A61K 31/554; A61K 31/21; A61K 31/34; A61K 31/517; A61K 31/167; A61K 31/165; A61K 31/138; A61K 31/4045; A61K 31/166; A61K 31/353; A61K 31/145; A61K 31/549

(52) U.S. Cl. CPC A61K 9/0034; A61K 31/549; A61K 31/41; A61K 31/4418; A61K 31/401; A61K 31/4178; A61K 31/4184; A61P 15/02; A61K 31/277; A61K 31/554; A61K 31/21; A61K 31/34; A61K 31/517; A61K 31/167; A61K 31/165; A61K 31/138; A61K 31/4045; A61K 31/166; A61K 31/353; A61K 31/145; A61K 31/50

(57) ABSTRACT

Methods are provided to prevent and to treat urogenital (e.g., urovaginal) atrophy syndrome of menopause by using a vasodilator such as an angiotensin receptor blocker, ACE inhibitor, or calcium channel blocker. More particularly, the methods do not employ orally administered vasodilators, but instead vasodilators that are administered through contact with the epidermis, e.g., in topical or other form suitable for contact with tissues to be treated.

### INCORPORATION BY REFERENCE TO RELATED APPLICATION

This application is a continuation of and claims the benefit of PCT Application No. PCT/US2019/033120, filed May 20, 2019, which claims priority to U.S. Provisional Application No. 62/675,144 filed May 22, 2018, U.S. Provisional Application No. 62/676,239, filed May 24, 2018, U.S. Provisional Application No. 62/675,776, filed May 24, 2018. Each of the aforementioned applications is incorporated by reference herein in its entirety, and each is hereby expressly made a part of this specification.

### FIELD OF THE INVENTION

Methods are provided to prevent and to treat urogenital (e.g., urovaginal) atrophy syndrome of menopause by using a vasodilator such as an angiotensin receptor blocker, ACE inhibitor, or calcium channel blocker. More particularly, the methods do not employ orally administered vasodilators, but instead vasodilators that are administered through contact with the epidermis, e.g., in topical or other form suitable for contact with tissues to be treated.

### BACKGROUND OF THE INVENTION

Since antiquity the aging of the urogenital organs in post menopausal period induced major life distresses and caused a significant and distressing reduction in the quality of life of post menopausal women. This seemingly unavoidable biological process induced a series of cellular, tissue and functional changes in women's urogenital tract that are now called the urogenital atrophy syndrome of menopause. Many scientific studies in the past 20 years discovered and detailed the biological and structural changes that take place in the vagina and bladder ureter areas in premenopausal and postmenopausal period, that every middle aged woman is familiar with: the distressing symptoms that such changes induce.

A recent international study explores the prevalence and detailed the spectrum of symptoms of the urogenital syndrome of menopause. The genital symptoms were: vaginal dryness, vaginal itching, vaginal burnings, vaginal pressure, vaginal spotting, vaginal discharge, and rarely vaginal bleeding. Dyspareunia or pain during sexual activity was found to be a cardinal and particularly distressing symptom of the urogenital atrophy syndrome of menopause. Other symptoms such as sexual dysfunction and post coital bleeding were demonstrated by this large international study as well. The spectrum of symptoms of the bladder and urethral areas caused by estrogen loss of menopause include: dysuria, urinary frequency, urethra discomfort, urethral pressure and frequent urinary infection. The biological cause of this distressing syndrome was discovered more than 80 years ago. This is due to the complete cessation of estrogen production by the ovaries imposed by the menopause process. Hormonal replacement therapy (HRT) became the therapeutic answers.

Between 1950 to 1990 multiple clinical trials of oral hormone replacement therapy gave the impression that the use of estrogens alone or with progestins was an efficient and safe treatment for the urogenital atrophy syndrome of menopause. In 1990 the Women's Health Initiative trial was initiated by the U.S. National Institute of health. These were three clinical studies involving 27,347 post menopausal women aged 50-79 that were followed during active hormonal treatments. The first study entailed 5.6 years in estrogen plus progestin trial. The second study entailed 7.2 years estrogen alone study. The third study entailed an extended period in which there was no treatment for a total follow up of 13 years. The results of these sentinel studies appeared in over 117 different scientific and medical publications. The cardinal findings were: Heart disease: 80% increase in the risk of coronary heart disease in the first year. 18% increase over the entire treatment period. Breast cancer: 24% increase in breast cancer over the entire treatment period. Stroke and blood clots: increase stroke risk by one third over the entire treatment period. Illness and death: 12% increase over the entire treatment period. Dementia: probable increase in dementia to women above the age of 65. The impact of the Women's Health Initiative Studies was unprecedented. This brought nearly to a halt the use of oral hormone replacement treatment and has continue to produces till today, a persistent fear in women all over the world, towards all form of hormonal replacement therapy.

### SUMMARY OF THE INVENTION

In view of the limitations of the current methods of prevention of urogenital atrophy syndrome, a new method of prevention and treatment is desirable. Provided is a method of applying a pharmaceutical preparation in an effective amount of a vasodilator, directly to the epidermis of the affected areas to prevent the formation of urogenital atrophy syndrome of menopause. Such a vasodilator can be considered a contact vasodilator. This is in contract to vasodilators administered in oral form for systemic delivery, e.g., in the treatment of conditions such as high blood pressure. Vasodilators administered in oral form for systemic delivery for treatment of conditions such as high blood pressure do not exhibit an impact on symptoms of urogenital atrophy syndrome. It has been surprisingly learned that formulation of the vasodilator into a form for contact with an epidermis to be treated (e.g., vaginal epidermis) results in lasting physiological changes, e.g., structural changes to the epidermis or adjacent tissue that can last, e.g., for several months or longer.

Accordingly, in a generally applicable first aspect (i.e., independently combinable with any of the aspects or embodiments identified herein), a pharmaceutical composition for the treatment or prophylaxis of urogenital atrophy syndrome is provided, comprising: at least one vasodilator; and at least one pharmaceutical excipient.

In a generally applicable embodiment (i.e., independently combinable with any of the aspects or embodiments identified herein) of the first aspect, the vasodilator is a contact vasodilator.

In a generally applicable embodiment (i.e., independently combinable with any of the aspects or embodiments identified herein) of the first aspect, the contact vasodilator is a calcium channel blocker.

In a generally applicable embodiment (i.e., independently combinable with any of the aspects or embodiments identified herein) of the first aspect, the at least one calcium channel blocker is a dihydropyridine selected from the group consisting of nifedipine, isradipine, felodipine, amlodipine, nicardipine, and clevidipine.

In a generally applicable embodiment (i.e., independently combinable with any of the aspects or embodiments identified herein) of the first aspect, the at least one calcium channel blocker is a non dihydropyridine selected from the group consisting of verapamil and diltiazem.

In a generally applicable embodiment (i.e., independently combinable with any of the aspects or embodiments identified herein) of the first aspect, the contact vasodilator is an ACE inhibitor.

In a generally applicable embodiment (i.e., independently combinable with any of the aspects or embodiments identified herein) of the first aspect, the ACE inhibitor is selected from the group consisting of benazepril, captopril, enalapril, fosinopril, lisinopril, moexipril, perindopril, quinapril, ramipril, and trandolapril.

In a generally applicable embodiment (i.e., independently combinable with any of the aspects or embodiments identified herein) of the first aspect, the contact vasodilator is an angiotensin receptor blocker.

In a generally applicable embodiment (i.e., independently combinable with any of the aspects or embodiments identified herein) of the first aspect, the angiotensin receptor blocker is selected from the group consisting of azilsartan, candesartan, eprosartan, irbesartan, losartan, olmesartan, telmisartan, and valsartan.

In a generally applicable embodiment (i.e., independently combinable with any of the aspects or embodiments identified herein) of the first aspect, the pharmaceutical composition is in a form is selected from the group consisting of a cream, a lotion, an ointment, a gel base, a foam, a powder, an aerosol spray, an oil, a liquid, and a suspension.

In a generally applicable embodiment (i.e., independently combinable with any of the aspects or embodiments identified herein) of the first aspect, the pharmaceutical composition is in a non-topical form.

In a generally applicable embodiment (i.e., independently combinable with any of the aspects or embodiments identified herein) of the first aspect, the urogenital atrophy syndrome is associated with menopause or estrogen deprivation syndrome.

In a generally applicable embodiment (i.e., independently combinable with any of the aspects or embodiments identified herein) of the first aspect, the non-topical form is selected from the group consisting of a vaginal tablet, a vaginal ovule, a vaginal suppository, and a vaginal insert.

In a generally applicable embodiment (i.e., independently combinable with any of the aspects or embodiments identified herein) of the first aspect, the non-topical form is impregnated with the at least one vasodilator, wherein the non-topical form is adapted for long term release of the at least vasodilator to a vaginal epidermis.

In a generally applicable embodiment (i.e., independently combinable with any of the aspects or embodiments identified herein) of the first aspect, the pharmaceutical composition is formulated as a liquid or a suspension of the at least one vasodilator.

In a generally applicable embodiment (i.e., independently combinable with any of the aspects or embodiments identified herein) of the first aspect, the pharmaceutical composition is formulated as a tablet comprising the at least one vasodilator.

In a generally applicable embodiment (i.e., independently combinable with any of the aspects or embodiments identified herein) of the first aspect, the pharmaceutical composition is formulated as an ovule comprising the at least one vasodilator.

In a generally applicable embodiment (i.e., independently combinable with any of the aspects or embodiments identified herein) of the first aspect, the pharmaceutical composition is formulated as a suppository comprising the at least one vasodilator.

In a generally applicable embodiment (i.e., independently combinable with any of the aspects or embodiments identified herein) of the first aspect, the pharmaceutical composition is formulated as an insert device impregnated with the at least one vasodilator adapted for long term release of the at least one vasodilator locally to an epidermis of the vagina.

Accordingly, in a generally applicable second aspect (i.e., independently combinable with any of the aspects or embodiments identified herein), a method is provided for the treatment or prophylaxis of urogenital atrophy syndrome in a patient in need thereof, comprising: administering an effective amount of the pharmaceutical composition according to the first aspect or any of its embodiments, whereby a condition associated with urogenital atrophy syndrome associated with menopause or estrogens deprivation syndromes in patients is treated or prevented.

Any of the features of an embodiment of the first and second aspects is applicable to all aspects and embodiments identified herein. Moreover, any of the features of an embodiment of the first and second aspects is independently combinable, partly or wholly with other embodiments described herein in any way, e.g., one, two, or three or more embodiments may be combinable in whole or in part. Further, any of the features of an embodiment of the first and second aspects may be made optional to other aspects or embodiments.

### DETAILED DESCRIPTION

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), directly to the epidermis of the urogenital areas to prevent the formation of urogenital atrophy syndrome of menopause.

A method is provided of applying a pharmacological composition in an effective amount, of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), directly to the epidermis of the urogenital areas, to treat the symptoms of urogenital atrophy syndrome of menopause.

The pharmacological preparation can comprise a calcium channel blocker. The calcium channel blocker can be in a suitable nontoxic pharmacological carrier.

The pharmacological preparation can comprise an ACE inhibitor. The ACE inhibitor can be in a suitable nontoxic pharmacological carrier.

The pharmacological preparation can comprise an angiotensin receptor blocker. The angiotensin receptor blocker can be in a suitable nontoxic pharmacological carrier.

An effective amount for treatment of the urogenital syndrome is administered. An amount of calcium channel blocker that is suitable for treatment by absorption through the epidermis of the urogenital areas is administered.

An effective amount for treatment of the urogenital syndrome is administered. An amount of ACE inhibitor that is suitable for treatment by absorption through the epidermis of the urogenital areas is administered.

An effective amount for treatment of the urogenital syndrome is administered. An amount of angiotensin receptor blocker that is suitable for treatment by absorption through the epidermis of the urogenital areas is administered.

Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply to the epidermis, e.g., in urogenital areas, which produces biological changes.

There is a need for a method to prevent or treat the urogenital atrophy caused by menopause by increasing the blood supply to the urogenital epidermis and subcutaneous soft tissue that produces the biological changes that prevent or treat the urogenital syndrome. There is a need for a method to prevent or treat the urogenital atrophy caused by menopause by activating or improving the function of the Bartholin's glands (also called Bartholin glands or greater vestibular glands).

A method is provided for preventing urogenital atrophy syndrome of menopause (e.g., atrophy of the urogenital areas that causes the symptoms of urogenital atrophy syndrome of menopause) by direct application to the urogenital epidermis of a vasodilator to prevent the formation and symptomatology of urogenital atrophy syndrome of menopause.

Also, treatment is provided of urogenital atrophy syndrome not caused by menopause, such as but not limited to, premature ovarian failure, oophorectomy, medications that block the estrogen activity, pituitary failure, Sheehan Syndrome, and medications that block the activity of pituitary stimulating hormones.

In certain embodiments, the vasodilator may be applied directly to the urogenital epidermis of peri or post menopausal women before symptoms arise, to prevent the urogenital atrophy syndrome, for example, in a woman who had the cessation of menses, but before the onset of the urogenital atrophy symptom.

In another embodiment, the vasodilators (e.g., calcium channel blockers, ACE inhibitors, angiotensin receptor blockers, nitrates, alpha blockers, beta blockers, hydralazine, and/or angiotensin receptor-neprilysin inhibitors) may be applied directly to the urogenital epidermis to treat and heal the symptoms of urogenital atrophy syndrome of menopause. The vasodilators (e.g., calcium channel blockers, ACE inhibitors, angiotensin receptor blockers, nitrates, alpha blockers, beta blockers, hydralazine, and/or angiotensin receptor-neprilysin inhibitors) may be applied even after the urogenital atrophy syndrome of menopause has dissipated and the urogenital epidermis has healed, to prevent the recurrence of urogenital atrophy syndrome of menopause.

Calcium channel blockers are a new class of pharmaceutical drugs that disrupt the entry of calcium molecules through the L type voltage operated channels to cardiac muscle and blood vessels cells. The blockage of calcium entry causes the relief of arterial spasm.

Currently there are 70 pharmaceutical patented calcium channel blocker drugs that use this property to treat hypertension, angina pectoris and cardiac arrhythmia. The clinical indication for the therapeutic use of calcium channel blockers was therefore limited, until now, to the field of cardiovascular diseases only.

Calcium channel blockers were extensively studied but their ability to prevent and or to treat urogenital atrophy syndrome of menopause remained heretofore unknown.

Accordingly, new uses are provided of topical calcium channel blockers for application to the urogenital epidermis and subcutaneous connective tissue. The new use may be used for the prevention or treatment of urogenital atrophy syndrome, associated with menopause or other syndromes associated with estrogen deprivation. No trial of topical calcium channel blockers for the prevention or treatment of urogenital atrophy syndrome of menopause has heretofore been published.

Contact calcium channel blockers are a part of contact-vasodilators, a new class of medication. The use is provided of contact neo-vasodilators such as Nifedipine, a known calcium channel blocker used in the treatment of hypertension, for the prevention and treatment urogenital atrophy syndrome of menopause.

Nifedipine, Amlodipine, Felodipine, Isradipine, Nicardipine, Nisoldipine and Clevidipine are in a class of dihydropyridines calcium channel blockers. Verapamil and Diltiazem are non-dihydropyridines calcium channel blockers. When applied topically these are very effective drugs for the treatment or prevention of urogenital atrophy syndrome caused by menopause, or urogenital atrophy syndrome associated with estrogen deprivation.

Inhibitors of angiotensin converting enzyme (ACE) can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik).

Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention or treatment of the urogenital atrophy associated with menopause or estrogen deprivation syndrome.

The spectrum of the genital atrophy syndrome includes symptoms including, but not limited to, one or more of dyspareunia (pain during sexual activity), vaginal dryness, vaginal itching, vaginal burning, vaginal pressure, vaginal spotting, vaginal discharge and vaginal bleeding associated with menopause or estrogen deprivation syndrome. The spectrum of urethral-bladder atrophy associated with menopause or estrogens deprivation syndrome includes, but is not limited to, one or more symptoms such as dysuria, urinary frequency urethral discomfort, urethral pressure and frequent urinary tract infections.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a topical form, such as, but not limited to, a cream, ointment, gel base, foam, powder, spray, oil, liquid preparation or suspension, to the vaginal epidermis can be employed to treat or prevent the symptoms of urogenital atrophy syndrome associated with menopause or estrogen deprivation syndrome. Other unit dosage forms include but are not limited to vaginal tablet, vaginal ovule, vaginal suppository, or vaginal insert impregnated with vasodilators (e.g., calcium channel blockers, ACE inhibitors, angiotensin receptor blockers, nitrates, alpha blockers, beta blockers, hydralazine, and/or angiotensin receptor-neprilysin inhibitors) for long term release.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable non toxic pharmaceutical carrier. The pharmaceutical composition in administered in an amount effective for treating urogenital atrophy syndrome associated with menopause or estrogen deprivation syndrome, e.g., an amount suitable for treatment by epidermal absorption, e.g., vaginal epidermis absorption.

Urogenital atrophy syndrome, associated symptoms, and treatment thereof are described in the following references, each of which is incorporated by reference herein in its entirety and each of which is hereby made a part of this specification: Gandhi J, Chen A, Dagur G, Suh Y, Smith N, Cali B, Khan S A. Genitourinary syndrome of menopause: an overview of clinical manifestations, pathophysiology, etiology, evaluation, and management. American journal of obstetrics and gynecology. 2016 Dec. 1; 215(6):704-11; Stuenkel C A, Davis S R, Gompel A, Lumsden M A, Murad M H, Pinkerton J V, Santen R J. Treatment of symptoms of the menopause: an endocrine society clinical practice guideline. The Journal of Clinical Endocrinology & Metabolism. 2015 Nov. 1; 100(11):3975-4011; American College of Obstetricians and Gynecologists. ACOG Practice Bulletin No. 141: management of menopausal symptoms. Obstet Gynecol. 2014; 123:202-16; Bachmann G, Santen R J. Treatment of genitourinary syndrome of menopause (vulvovaginal atrophy). UpToDate online database. Available at www.uptodate.com (with subscription). Accessed October. 2016; Leiblum S, Bachmann G, Kemmann E, Colburn D, Swartzman L. Vaginal atrophy in the postmenopausal woman: the importance of sexual activity and hormones. Jama. 1983 Apr. 22; 249(16):2195-8; Barlow D H, Cardozo L D, Francis R M, Griffin M, Hart D M, Stephens E, Sturdee D W. Urogenital ageing and its effect on sexual health in older British women. BJOG: An International Journal of Obstetrics & Gynaecology. 1997 January; 104(1):87-91; Abernethy D R, Schwartz J B. Calcium-antagonist drugs. New England Journal of Medicine. 1999 Nov. 4; 341(19):1447-57; Godfraind T. Discovery and development of calcium channel blockers. Frontiers in pharmacology. 2017 May 29; 8:286; Woods N F, Mitchell E S. Symptoms during the perimenopause: prevalence, severity, trajectory, and significance in women's lives. The American journal of medicine. 2005 Dec. 19; 118(12):14-24; Gold E B, Colvin A, Avis N, Bromberger J, Greendale G A, Powell L, Sternfeld B, Matthews K. Longitudinal analysis of the association between vasomotor symptoms and race/ethnicity across the menopausal transition: study of women's health across the nation. American journal of public health. 2006 July; 96(7):1226-35; National Institutes of Health. National Institutes of Health State-of-the-Science Conference statement: management of menopause-related symptoms. Annals of Internal Medicine. 2005 Jun. 21; 142(12):1003; Dennerstein L, Dudley E C, Hopper J L, Guthrie J R, Burger H G. A prospective population-based study of menopausal symptoms. Obstetrics & Gynecology. 2000 Aug. 23; 96(3):351-8; Harlow S D, Gass M, Hall J E, Lobo R, Maki P, Rebar R W, Sherman S, Sluss P M, de Villiers T J, STRAW+ 10 Collaborative Group. Executive summary of the Stages of Reproductive Aging Workshop+ 10: addressing the unfinished agenda of staging 1; 97(4):1159-68; Tepper P G, Brooks M M, Randolph Jr J F, Crawford S L, El Khoudary S R, Gold E B, Lasley B L, Jones B, Joffe H, Hess R, Avis N E. Characterizing the trajectories of vasomotor symptoms across the menopausal transition. Menopause (New York, N.Y.). 2016 October; 23(10):1067; Thurston R C, Joffe H. Vasomotor symptoms and menopause: findings from the Study of Women's Health across the Nation. Obstetrics and Gynecology Clinics. 2011 Sep. 1; 38(3):489-501; Randolph Jr J F, Sowers M, Bondarenko I, Gold E B, Greendale G A, Bromberger J T, Brockwell S E, Matthews K A. The relationship of longitudinal change in reproductive hormones and vasomotor symptoms during the menopausal transition. The Journal of Clinical Endocrinology & Metabolism. 2005 Nov. 1; 90(11):6106-12; Nappi R E, Kokot-Kierepa M. Women's voices in the menopause: results from an international survey on vaginal atrophy. Maturitas. 2010 Nov. 1; 67(3):233-8; Portman D J, Gass M L, Vulvovaginal Atrophy Terminology Consensus Conference Panel. Genitourinary syndrome of menopause: new terminology for vulvovaginal atrophy from the International Society for the Study of Women's Sexual Health and the North American Menopause Society. Climacteric. 2014 Oct. 1; 17(5):557-63; Johnston S L, Farrell S A, Bouchard C, Farrell S A, Beckerson L A, Comeau M. The detection and management of vaginal atrophy. J Obstet Gynaecol Can. 2004 May; 26(5):503-15; Mitchell C M, Reed S D, Diem S, Larson J C, Newton K M, Ensrud K E, LaCroix A Z, Caan B, Guthrie K A. Efficacy of vaginal estradiol or vaginal moisturizer vs placebo for treating postmenopausal vulvovaginal symptoms: a randomized clinical trial. JAMA internal medicine. 2018 May 1; 178(5):681-90; Lethaby A, Ayeleke R O, Roberts H. Local oestrogen for vaginal atrophy in postmenopausal women. Cochrane Database of Systematic Reviews. 2016(8); Barnabei V M, Cochrane B B, Aragaki A K, Nygaard I, Williams R S, McGovern P G, Young R L, Wells E C, O'Sullivan M J, Chen B, Schenken R. Menopausal symptoms and treatment-related effects of estrogen and progestin in the Women's Health Initiative. Obstetrics & Gynecology. 2005 May 1; 105(5):1063-73; Marjoribanks J, Farquhar C, Roberts H, Lethaby A. Long term hormone therapy for perimenopausal and postmenopausal women. Cochrane Database of Systematic Reviews. 2012(7); Pandit L, Ouslander J G. Postmenopausal vaginal atrophy and atrophic vaginitis. The American journal of the medical sciences. 1997 Oct. 1; 314(4):228-31; Beard M K. Atrophic vaginitis: can it be prevented as well as treated?. Postgraduate medicine. 1992 May 1; 91(6):257-60; J S, Richter H E, Walter L C, Thom D, Stewart A L. The day-to-day impact of vaginal aging questionnaire: A multidimensional measure of the impact of vaginal symptoms on functioning and well-being in postmenopausal women. Menopause (New York, N.Y.). 2015 February; 22(2):144; Randolph Jr J F, Crawford S, Dennerstein L, Cain K, Harlow S D, Little R, Mitchell E S, Nan B, Taffe J, Yosef M. The value of follicle-stimulating hormone concentration and clinical findings as markers of the late menopausal transition. The Journal of Clinical Endocrinology & Metabolism. 2006 Aug. 1; 91(8):3034-40; Hall J E. Neuroendocrine physiology of the early and late menopause. Endocrinology and Metabolism Clinics. 2004 Dec. 1; 33(4):637-59; Santen R J, Allred D C, Ardoin S P, Archer D F, Boyd N, Braunstein G D, Burger H G, Colditz G A, Davis S R, Gambacciani M, Gower B A. Postmenopausal hormone therapy: an Endocrine Society scientific statement. The Journal of Clinical Endocrinology & Metabolism. 2010 Jul. 1; 95(7_supplement_1):s1-66; and Chlebowski R T, Cirillo D J, Eaton C B, Stefanick M L, Pettinger M, Carbone L D, Johnson K C, Simon M S, Woods N F, Wactawski-Wende J. Estrogen alone and joint symptoms in the Women's Health Initiative randomized trial. Menopause (New York, N.Y.). 2013 June; 20(6); Genitourinary syndrome of menopause: an overview of clinical manifestations, pathophysiology, etiology, and management. Am. Obstet. Gynecol. 2016; 215:704; Treatment of symptoms of the Menopause: an endocrine society clinical practice guideline. J. Clin. Endocrinol. Metab. 2015; 100:3975; ACOG Practice Bulletin No 141: Management of Menopausal symptoms. Obstet. gynecol. 2014; 123:202; Dyspareunia and Vaginal dryness SRM 2008; 6:18; Vaginal atrophy in the postmenopausal women. The importance of sexual activity and hormones. JAMA 1983; 249:2195; Urogenital ageing and its effects on sexual health in older British women. Br. J. Obstet. Gynecol. 1997:104:87; Genitourinary Syndrome of Menopause: An Overview of Clinical Manifestations, Pathophysiology, Etiology, and Management. Am. Obstet. Gynecol. 2016; 215:704; Treatment of Symptoms of the Menopause: An Endocrine Society Clinical Practice Guideline. J. Clin. Endocrinol. Metab. 2015; 100:3975; ACOG Practice Bulletin No 141: Management of Menopausal Symptoms Obstet. gynecol. 2014; 123:202; Dyspareunia and Vaginal Dryness SRM 2008:6:18; Vaginal atrophy in the Post Menopausal women. The importance of Sexual Activity and Hormones. JAMA 1983; 249:2195; Urogenital Ageing and Its Effects on Sexual Health in older British women. Br. J. Obstet. Gynecol. 1997:104:87; Angiotensin receptors antagonists Lancet 1997 349 1255; Angiotensin receptor blockers for heart failure Cochran's Database Sys, Rev 2012 CD0003040; Angiotensin Converting Enzymes inhibitors and Angiotensin Receptor blockers in Myocardial Infarction Patients with Renal Dysfunction Jam Coll Cardiol 2016.

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies, and associated methods for treatment of urogenital atrophy and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem L A, Tiazac), felodipine (Plendil), isradipine (Dynacirc), nifedipine (Adalat, Procardia), nicardipine (Cardene), nimodipine (Nimotop), nisoldipine (Sular), verapamil (Covera-H S, Verelan P M, Calan), verapamil, diltiazem and nicardipine (Cardene IV). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for topical administration, e.g., to vaginal epidermis, however other routes of administration are also considered that involve contact of the vasodilator to the tissue to be treated.

The pharmaceutical compositions for treatment of urogenital atrophy can further comprise other pharmaceutically active ingredients (e.g., estrogen), or can be administered in conjunction with a conventional treatment for urogenital atrophy, e.g., estrogen or hormone replacement therapy (ERT-HRT), a vaginal moisturizer (K-Y Liquibeads, Replens, Sliquid, etc.), or a water-based lubricant (Astroglide, K-Y Jelly, Sliquid, etc.). The treatment can be administered in conjunction with other therapies, e.g., administration of ospemifene (Osphena), prasterone (Intrarosa), systemic estrogen therapy, vaginal dilators, topical lidocaine, or any other conventional treatment employed in conjunction with urogenital (e.g., vaginal) atrophy.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. calcium channel blockers) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., calcium channel blockers, ACE inhibitors, angiotensin receptor blockers, nitrates, alpha blockers, beta blockers, hydralazine, and/or angiotensin receptor-neprilysin inhibitors) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment of urogenital atrophy or associated symptoms. The compositions are suitable for use in any patient where treatment of urogenital atrophy is desirable, e.g., premenopausal, perimenopausal, or postmenopausal women.

The compositions can also be employed in the connection with mucous membranes, in particular the vaginal mucosa. When applied to the vaginal mucosa, a vaginal applicator can be employed as are commercially available. Suitable applicators can be in a form of a pre-filled syringe, a tube attached to a prefilled squeezable reservoir, a prepackaged wand including a preselected amount of composition, or a universal vaginal applicator including perforations along its length for dispensing the composition through the perforations.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for application to genital tissue or mucous membranes. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid skin irritation associated with certain preservatives.

To facilitate application, the composition may be provided as an ointment, an oil, a lotion, a paste, a powder, a gel, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied to the dermis, or to mucous membranes.

Methods of using topical vasodilator formulations are provided. The compositions may also be applied to treat urogenital atrophy and/or associated symptoms.

Some embodiments include administering vasodilator (e.g., a calcium channel blocker, ACE inhibitor and/or angiotensin receptor blocker) compositions provided herein in topical formulations; however, other routes of administration are also contemplated (e.g., mucosal, subdermal, oral, or the like). Contemplated routes of administration include but are not limited to topical, mucosal, and subcutaneous. Suitable liquid forms include suspensions, emulsions, solutions, and the like. Unit dosage forms can also be provided, e.g., individual packets with a premeasured amount of the formulation, configured for administration to the genital tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and other formulations typically comprise from about 0.001 wt. % or less to about 50 wt. % or more of active ingredient, such as the vasodilator (e.g., a calcium channel blocker, ACE inhibitor and/or angiotensin receptor blocker), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration can include transdermal patches, ointments, lotions, creams, gels, drops, sprays, liquids, aerosols, and powders. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the tissue surface, liquid in the container is transferred in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical compositions. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to skin or mucous membranes.

Penetration enhancers can be employed to enhance penetration of the vasodilator into tissue, and to provide a silky feel to formulations. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the vasodilator as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the skin or mucous membranes.

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilator compositions as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilator compositions as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

### Kits for Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator in a suitable topical formulation, and instructions for administering the composition to a subject. The kit can optionally also contain one or more additional therapeutic or other agents, e.g., estrogen or a hormone replacement therapy. For example, a kit containing a vasodilator in topical form can be provided along with other agents such as moisturizers or lubricants. The kit may contain the vasodilator in bulk form, or can contain separate doses of the vasodilator for serial or sequential administration. The kit can optionally contain one or more diagnostic tools, administration tools, and/or instructions for use. The kit can contain suitable delivery devices, such as, syringes, pump dispensers, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation, in addition to the vasodilator, can contain other ingredients.

While topical administration of the vasodilator disclosed herein can advantageously be employed, in certain embodiments other routes of administration are also contemplated.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, a powder, as granules, as a solution, as a suspension in an aqueous liquid, as a non-aqueous liquid, as an oil-in-water emulsion, or as a water-in-oil liquid emulsion, similar to the topical formulations described elsewhere herein, but using components suitable for human consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation in skin tissue. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition, e.g., with estrogen or a hormone replacement therapy. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include hormones, anti-infective agents, anti-inflammatory agents, anesthetics, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

### EXAMPLES

The following prophetic examples are of cases that address the painful experiences associated with postmenopause during intimate acts by three different couples. The examples also describe a new, safe, simple and until now, an unknown pharmaceutical solution to the worldwide problem of post menopausal dyspareunia syndrome: the novel use of vasodilators such as calcium channel blockers, ACE inhibitors, and angiotensin receptor blockers. Vasodilators are not analgesics, but they resolve the pain associated with post menopausal intimate activity. Vasodilators are not lubricants but they resolve the post menopausal vaginal dryness. Vasodilators are not hormones, but they resume the glandular activity of the estrogen dependent atrophic vaginal glands. Vasodilators are among the safest, widely used medication in the world. These cases unveil the first pharmaceutical drugs that at least temporarily reverse the aging process as manifested in improvements of three age related menopausal atrophic processes. They are: the shrinking of the introits, and reversal of the introitus diameter shrinking, to post adolescence size; reversal of the atrophic and rigid contraction of menopausal vaginal canal, and recovery of the elastic properties of the vaginal tube characteristic of younger adults; and resumption of the lubricating activity of the atrophic vaginal glands, without the resumption of menses or ovarian activity. All anatomical and physiological vaginal changes are construed from the different accounts of the participant experiences of pain and subsequent pain resolution. These involve pain onset, pain sites, pain duration, pain quality and pain intensity and their respective resolutions. All the anatomically perceived changes are derived from detailed perceptions of the individuals involved. The resumption of the secretory atrophic glandular activity is based on direct observation.

### Example 1

A couple share their frustration of their intimate activity experiences during the pre menopausal years. A few months after the beginning of irregular menses, they are surprised by sudden onset of severe and sharp pain during intimate activity. This is associated with an unexpected narrowing of vaginal introits diameter. Vaginal dryness is first experienced. The initial event leads to abrupt withdrawal of activity. Similar events occur in the following month, and with increased frequency over the next two years. These lead to refusal to engage in intimate activity for prolong periods, initially measured in weeks and later in months. By the time menses completely ends, a halt of intimate activity is already established. Pain is blamed on the following age related changes: a contracted introits; severe vaginal dryness; a significant reduction in vaginal canal diameter; and Loss of elasticity. All these become major physical and psychological issues. The patient declines her gynecologist's suggestion of hormone-based drugs because on their known risks. A negative perception becomes attached to what was previously a rewarding sexual activity. By the end of the first two premenopausal years, intimate activity is perceived as a pain producing act that is best avoided. Little do they know that such experiences are felt by millions couples worldwide. In the following three years they attempt to solve the issue by use of different lubricants, but slowly abandon this approach. Lubricants produce a messy, reduced, and less gratifying reward as they only partially correct the anatomical changes. Two years after the appearance of irregular menses and four years after their complete cessation, even rare sexual activity stops. The three described anatomical changes become a permanent fact.

In the beginning of the seventh post menopausal year, a daily intra vaginal use of the content of a nifedipine capsule is initiated. Nifedipine is a known blood pressure medication. Within two weeks they report that their detailed difficulties significantly resolved. A complete resolution is achieved by the end of the first month. The benefit lasts to the end of the drug supply. These benefits include the following: increase in the diameter of the introitus; increase in the perceived elasticity and stretchability of the vaginal tube; and resumption of the secretory activity of the vaginal glands, caused by the resumption of the natural vaginal lubrication.

### Example 2

This case involves the use of a different calcium channel blocker. During the premenopausal years, this couple experiences similar difficulties as the first couple described in Example 1, and attempts several available remedies. By the end of the third premenopausal year they are similarly frustrated. Their experiences detail the same age related changes, and have similar impact on their intimate activity. Even before the complete cessation of menses, all their intimate activity ceases. The couple's perception is that body changes and their impact on the intimate act are the physiological normality of the aging process. Three years after the onset of premenopausal changes and two years after the complete cessation of periods, they become aware of first couple's experience with Nifedipine (Example 1). What follows is a friendly request for similar help. This time, Amlodipine—a different calcium channel blocker—is offered. The advice includes holding resumption of intimate activity until after three weeks of Amlodipene use. In total, the perceived results following drug use are similar to the first couple's experience. One particular detail is significant—the observation of visible vaginal secretions outside the pelvic tube. They consider it significant because such event does not occur in the previous three years. They confirm what was, until then, only assumed—that the calcium channel blocker causes resumption of vaginal glandular activity in the absence of ovarian hormones.

### Example 3

The third case testimony also contributes important data. The pre drug experiences and the drug period experiences of the couple mimic the experiences of the previous couples (Example 1 and Example 2). They contribute new and significant observation: that the benefits of the calcium channel blocker lasted at least two months beyond its last administration. This raises the possibility that at least for those 8 weeks, the aging process (as manifested by the couples' experiences) is not merely halted but reversed. The potency may last even longer. Such beneficial biological process is heretofore never described or published.

### Example 4

A couple share their frustration of their intimate activity experiences during the pre menopausal years. A few months after the beginning of irregular menses, they are surprised by sudden onset of severe and sharp pain during intimate activity. This is associated with an unexpected narrowing of vaginal introits diameter. Vaginal dryness is first experienced. The initial event leads to abrupt withdrawal of activity. Similar events occur in the following month, and with increased frequency over the next two years. These lead to refusal to engage in intimate activity for prolong periods, initially measured in weeks and later in months. By the time menses completely ends, a halt of intimate activity is already established. Pain is blamed on the following age related changes: a contracted introits; severe vaginal dryness; a significant reduction in vaginal canal diameter; and loss of elasticity. All these become major physical and psychological issues. The patient declines her gynecologist's suggestion of hormone-based drugs because on their known risks. A negative perception becomes attached to what was previously a rewarding sexual activity. By the end of the first two premenopausal years, intimate activity is perceived as a pain producing act that is best avoided. Little do they know that such experiences are felt by millions couples worldwide. In the following three years they attempt to solve the issue by use of different lubricants, but slowly abandon this approach. Lubricants produce a messy, reduced, and less gratifying reward as they only partially correct the anatomical changes. Two years after the appearance of irregular menses and four years after their complete cessation, even rare sexual activity stops. The three described anatomical changes become a permanent fact.

In the beginning of the seventh post menopausal year, a daily intra vaginal use of the content of an enalapril capsule is initiated. Enalapril is a known blood pressure medication. Within two weeks they report that their detailed difficulties significantly resolved. A complete resolution is achieved by the end of the first month. The benefit lasts to the end of the drug supply. These benefits include the following: increase in the diameter of the introitus; increase in the perceived elasticity and stretchability of the vaginal tube; and resumption of the secretory activity of the vaginal glands, caused by the resumption of the natural vaginal lubrication.

### Example 5

This case involves the use of a different ACE inhibitor. During the premenopausal years, this couple experiences similar difficulties as the first couple described in Example 1, and attempts several available remedies. By the end of the third premenopausal year they are similarly frustrated. Their experiences detail the same age related changes, and have similar impact on their intimate activity. Even before the complete cessation of menses, all their intimate activity ceases. The couple's perception is that body changes and their impact on the intimate act are the physiological normality of the aging process. Three years after the onset of premenopausal changes and two years after the complete cessation of periods, they become aware of first couple's experience with enalapril (Example 4). What follows is a friendly request for similar help. This time, lisinopril—a different ACE inhibitor—is offered. The advice includes holding resumption of intimate activity until after three weeks of lisinopril use. In total, the perceived results following drug use are similar to the first couple's experience. One particular detail is significant—the observation of visible vaginal secretions outside the pelvic tube. They consider it significant because such event does not occur in the previous three years. They confirm what was, until then, only assumed—that the ACE inhibitor causes resumption of vaginal glandular activity in the absence of ovarian hormones.

### Example 6

The third case testimony also contributes important data. The pre drug experiences and the drug period experiences of the couple mimic the experiences of the previous couples (Example 4 and Example 5). They contribute new and significant observation: that the benefits of the ACE inhibitor lasted at least two months beyond its last administration. This raises the possibility that at least for those 8 weeks, the aging process (as manifested by the couples' experiences) is not merely halted but reversed. The potency may last even longer. Such beneficial biological process is heretofore never described or published.

### Example 7

A couple share their frustration of their intimate activity experiences during the pre menopausal years. A few months after the beginning of irregular menses, they are surprised by sudden onset of severe and sharp pain during intimate activity. This is associated with an unexpected narrowing of vaginal introits diameter. Vaginal dryness is first experienced. The initial event leads to abrupt withdrawal of activity. Similar events occur in the following month, and with increased frequency over the next two years. These lead to refusal to engage in intimate activity for prolong periods, initially measured in weeks and later in months. By the time menses completely ends, a halt of intimate activity is already established. Pain is blamed on the following age related changes: a contracted introits; severe vaginal dryness; a significant reduction in vaginal canal diameter; and loss of elasticity. All these become major physical and psychological issues. The patient declines her gynecologist's suggestion of hormone-based drugs because on their known risks. A negative perception becomes attached to what was previously a rewarding sexual activity. By the end of the first two premenopausal years, intimate activity is perceived as a pain producing act that is best avoided. Little do they know that such experiences are felt by millions couples worldwide. In the following three years they attempt to solve the issue by use of different lubricants, but slowly abandon this approach. Lubricants produce a messy, reduced, and less gratifying reward as they only partially correct the anatomical changes. Two years after the appearance of irregular menses and four years after their complete cessation, even rare sexual activity stops. The three described anatomical changes become a permanent fact.

In the beginning of the seventh post menopausal year, a daily intra vaginal use of the content of a losartan capsule is initiated. Losartan is a known blood pressure medication. Within two weeks they report that their detailed difficulties significantly resolved. A complete resolution is achieved by the end of the first month. The benefit lasts to the end of the drug supply. These benefits include the following: increase in the diameter of the introitus; increase in the perceived elasticity and stretchability of the vaginal tube; and resumption of the secretory activity of the vaginal glands, caused by the resumption of the natural vaginal lubrication.

### Example 8

This case involves the use of a different angiotensin receptor blocker. During the premenopausal years, this couple experiences similar difficulties as the first couple described in Example 1, and attempts several available remedies. By the end of the third premenopausal year they are similarly frustrated. Their experiences detail the same age related changes, and have similar impact on their intimate activity. Even before the complete cessation of menses, all their intimate activity ceases. The couple's perception is that body changes and their impact on the intimate act are the physiological normality of the aging process. Three years after the onset of premenopausal changes and two years after the complete cessation of periods, they become aware of first couple's experience with enalapril (Example 4). What follows is a friendly request for similar help. This time, irbesartan—a different angiotensin receptor blocker—is offered. The advice includes holding resumption of intimate activity until after three weeks of irbesartan use. In total, the perceived results following drug use are similar to the first couple's experience. One particular detail is significant—the observation of visible vaginal secretions outside the pelvic tube. They consider it significant because such event does not occur in the previous three years. They confirm what was, until then, only assumed—that the angiotensin receptor blocker causes resumption of vaginal glandular activity in the absence of ovarian hormones.

### Example 9

The third case testimony also contributes important data. The pre drug experiences and the drug period experiences of the couple mimic the experiences of the previous couples (Example 4 and Example 5). They contribute new and significant observation: that the benefits of the angiotensin receptor blocker lasted at least two months beyond its last administration. This raises the possibility that at least for those 8 weeks, the aging process (as manifested by the couples' experiences) is not merely halted but reversed. The potency may last even longer. Such beneficial biological process is heretofore never described or published.

### Exemplary Compositions, Methods and Uses

Pharmaceutical Composition 1: A pharmaceutical composition for the treatment or prophylaxis of urogenital atrophy syndrome, comprising: at least one vasodilator; and at least one pharmaceutical excipient.

Pharmaceutical Composition 2: Pharmaceutical Composition 1, for the treatment of urogenital atrophy syndrome, optionally associated with menopause or estrogen deprivation syndrome.

Pharmaceutical Composition 3: Pharmaceutical Composition 1, for the prophylaxis of urogenital atrophy syndrome, optionally associated with menopause or estrogen deprivation syndrome.

Pharmaceutical Composition 4: Any one of Pharmaceutical Compositions 1 through 3, in a form adapted for topical administration to an epidermis, optionally a vaginal epidermis.

Pharmaceutical Composition 5: Pharmaceutical Composition 4, wherein the form is selected from the group consisting of a cream, a lotion, an ointment, a gel base, a foam, a powder, an aerosol spray, an oil, a liquid, and a suspension.

Pharmaceutical Composition 6: Any one of Pharmaceutical Compositions 1 through 3, in a non-topical form, wherein the vasodilator is a contact vasodilator.

Pharmaceutical Composition 7: Pharmaceutical Composition 6, wherein the non-topical form is selected from the group consisting of a vaginal tablet, a vaginal ovule, a vaginal suppository, and a vaginal insert, wherein the non-topical form is impregnated with the at least one vasodilator, wherein the non-topical form is adapted for long term release of the at least one vasodilator to a vaginal epidermis.

Pharmaceutical Composition 8: Any one of Pharmaceutical Compositions 1 through 3, formulated as a liquid or a suspension of the at least one vasodilator, wherein the vasodilator is a contact vasodilator.

Pharmaceutical Composition 9: Any one of Pharmaceutical Compositions 1 through 3, formulated as a tablet comprising the at least one vasodilator, wherein the vasodilator is a contact vasodilator.

Pharmaceutical Composition 10: Any one of Pharmaceutical Compositions 1 through 3, formulated as an ovule comprising the at least one vasodilator, wherein the vasodilator is a contact vasodilator.

Pharmaceutical Composition 11: Any one of Pharmaceutical Compositions 1 through 3, formulated as a suppository comprising the at least one vasodilator, wherein the vasodilator is a contact vasodilator.

Pharmaceutical Composition 12: Any one of Pharmaceutical Compositions 1 through 3, formulated as an insert device impregnated with the at least one vasodilator adapted for long term release of the at least one vasodilator locally to an epidermis of the vagina.

Pharmaceutical Composition 13: Any one of Pharmaceutical Compositions 1 through 12, wherein the vasodilator is a calcium channel blocker.

Pharmaceutical Composition 14: Any one of Pharmaceutical Compositions 13, wherein the at least one calcium channel blocker is a dihydropyridine selected from the group consisting of nifedipine, isradipine, felodipine, amlodipine, nicardipine, and clevidipine.

Pharmaceutical Composition 15: Pharmaceutical Composition 13, wherein the at least one calcium channel blocker is a non dihydropyridine selected from the group consisting of verapamil and diltiazem.

Pharmaceutical Composition 16: Any one of Pharmaceutical Compositions 1 through 12, wherein the vasodilator is an ACE inhibitor.

Pharmaceutical Composition 17: Pharmaceutical Composition 16, wherein the ACE inhibitor is selected from the group consisting of benazepril, captopril, enalapril, fosinopril, lisinopril, moexipril, perindopril, quinapril, ramipril, and trandolapril.

Pharmaceutical Composition 18: Any one of Pharmaceutical Compositions 1 through 12, wherein the vasodilator is an angiotensin receptor blocker.

Pharmaceutical Composition 19: Pharmaceutical Composition 18, wherein the angiotensin receptor blocker is selected from the group consisting of azilsartan, candesartan, eprosartan, irbesartan, losartan, olmesartan, telmisartan, and valsartan.

Pharmaceutical Composition 20: Any one of Pharmaceutical Compositions 1 through 12, wherein the vasodilator is a nitrate.

Pharmaceutical Composition 21: Pharmaceutical Composition 20, wherein the nitrate is selected from the group consisting of nitroglycerin, isosorbide mononitrate and isosorbide dinitrate.

Pharmaceutical Composition 22: Any one of Pharmaceutical Compositions 1 through 12, wherein the vasodilator is an alpha blocker.

Pharmaceutical Composition 23: Pharmaceutical Composition 22, wherein the alpha blocker is selected from the group consisting of doxazosin, prazosin, and terazosin.

Pharmaceutical Composition 24: Any one of Pharmaceutical Compositions 1 through 12, wherein the vasodilator is a beta blocker.

Pharmaceutical Composition 25: Pharmaceutical Composition 24, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 26: Any one of Pharmaceutical Compositions 1 through 12, wherein the vasodilator is hydralazine.

Pharmaceutical Composition 27: Any one of Pharmaceutical Compositions 1 through 12, wherein the vasodilator is an angiotensin receptor-neprilysin inhibitor.

Pharmaceutical Composition 28: Pharmaceutical Composition 27, wherein the angiotensin receptor-neprilysin inhibitor is sacubitril/valsartan.

Method 29: A method for the treatment or prophylaxis of urogenital atrophy syndrome in a patient in need thereof, comprising: administering an effective amount of the pharmaceutical composition according to any one of Pharmaceutical Compositions 1 through 19 to a patient in need thereof, whereby a condition associated with urogenital atrophy syndrome, optionally associated with menopause or estrogen deprivation syndrome, is treated or prevented.

Method 30: Method 29, for the treatment of urogenital atrophy syndrome.

Method 31: Method 29, for the prophylaxis of urogenital atrophy syndrome.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound of Formula (I) as described herein or any compound having a structure described herein, and any aspect or embodiment of a composition, e.g., a pharmaceutical composition and/or a compound of Formula (I) or any compound having a structure described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition for the treatment or prophylaxis of urogenital atrophy syndrome, comprising:
at least one vasodilator; and
at least one pharmaceutical excipient.

2. The pharmaceutical composition of claim 1, formulated as an ovule comprising the at least one vasodilator, wherein the vasodilator is a contact vasodilator.

3. The pharmaceutical composition of claim 1, for the treatment of urogenital atrophy syndrome, optionally associated with menopause or estrogen deprivation syndrome.

4. The pharmaceutical composition of claim 1, in a form adapted for topical administration to an epidermis, optionally a vaginal epidermis.

5. The pharmaceutical composition of claim 1, in a non-topical form, wherein the vasodilator is a contact vasodilator.

6. The pharmaceutical composition of claim 5, wherein the non-topical form is selected from the group consisting of a vaginal tablet, a vaginal ovule, a vaginal suppository, and a vaginal insert, wherein the non-topical form is impregnated with the at least one vasodilator, wherein the non-topical form is adapted for long term release of the at least one vasodilator to a vaginal epidermis.

7. The pharmaceutical composition of claim 1, formulated as a liquid or a suspension of the at least one vasodilator, wherein the vasodilator is a contact vasodilator.

8. The pharmaceutical composition of claim 1, formulated as a tablet comprising the at least one vasodilator, wherein the vasodilator is a contact vasodilator.

9. The pharmaceutical composition of claim 1, wherein the vasodilator is a calcium channel blocker selected from the group consisting of nifedipine, isradipine, felodipine, amlodipine, nicardipine, and clevidipine.

10. The pharmaceutical composition of claim 1, wherein the vasodilator is a calcium channel blocker selected from the group consisting of verapamil and diltiazem.

11. The pharmaceutical composition of claim 1, wherein the vasodilator is an ACE inhibitor selected from the group consisting of benazepril, captopril, enalapril, fosinopril, lisinopril, moexipril, perindopril, quinapril, ramipril, and trandolapril.

12. The pharmaceutical composition of claim 1, wherein the vasodilator is an angiotensin receptor blocker selected from the group consisting of azilsartan, candesartan, eprosartan, irbesartan, losartan, olmesartan, telmisartan, and valsartan.

13. The pharmaceutical composition of claim 1, wherein the vasodilator is a nitrate selected from the group consisting of nitroglycerin, isosorbide mononitrate and isosorbide dinitrate.

14. The pharmaceutical composition of claim 1, wherein the vasodilator is an alpha blocker selected from the group consisting of doxazosin, prazosin, and terazosin.

15. The pharmaceutical composition of claim 1, wherein the vasodilator is a beta blocker selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

16. The pharmaceutical composition of claim 1, wherein the vasodilator is hydralazine.

17. The pharmaceutical composition of claim 1, wherein the vasodilator is an angiotensin receptor-neprilysin inhibitor.

18. The pharmaceutical composition of claim 17, wherein the angiotensin receptor-neprilysin inhibitor is sacubitril/valsartan.

19. A method for the treatment or prophylaxis of urogenital atrophy syndrome in a patient in need thereof, comprising:
administering an effective amount of the pharmaceutical composition according to claim 1 to a patient in need thereof,
whereby a condition associated with urogenital atrophy syndrome, optionally associated with menopause or estrogen deprivation syndrome, is treated or prevented.

20. The method of claim 19, for the treatment of urogenital atrophy syndrome.
